(19) United States
(12) Patent Application Publication
(10) Pub. No.: US 2016/0228292 A1
(43) Pub. Date: Aug. 11, 2016

(54) DEVICES AND METHODS TO FACILITATE EYE POSITIONING AND EYE DROP INSTALLATION

(71) Applicant: Peter MICHALOS, Southampton, NY (US)

(72) Inventor: Peter MICHALOS, Southampton, NY (US)

(21) Appl. No.: 14/696,447

(22) Filed: Apr. 26, 2015

Related U.S. Application Data
(60) Provisional application No. 62/072,434, filed on Oct. 30, 2014.

(51) Int. Cl. A61F 9/00; G02C 5/00; G02C 5/14; G02C 11/00; G02C 5/16

(52) U.S. Cl. CPC A61F 9/0026; G02C 11/00; G02C 5/16; G02C 5/146; G02C 5/008; G02C 2200/08

(57) ABSTRACT

A frame assembly, for wearing on the head of a person, includes left and right eye plates, each having an optical aperture and a liquid aperture. A temple includes a first post defining a first extremity and a second post defining a second extremity. The temple is subject to compression force such that a distance E between the first and second extremities is less that a distance S between holes on the frame front. The temple is resilient such that, after compression ceases, the distance E is greater than the distance S, thereby enabling the temple to be removably attachable to the frame front, without using a tool.

This Application claims the benefit of U.S. Provisional Patent Application 62/072,434 filed 30 Oct. 2014 of PETER MICHALOS for EYEDROP DELIVERY SYSTEM, the contents of which are herein incorporated by reference.

### BACKGROUND OF THE INVENTION

1. Field of the Invention

This invention relates generally to applying eye drops to human eyes and, more particularly, to applicators that facilitate application of eye drops.

2. Description of Related Art

Placement of eye drops can be challenging, especially for people having certain conditions such as Arthritis or Parkinson's disease. Some conventional methods of applying eye drops can result in misapplication of the eye drops and the risk of poking the eye and scratching the cornea.

### SUMMARY OF THE INVENTION

It is an object of the present invention to provide an eye drop applicator to a frame assembly having opaque eye plates with a liquid aperture and an optical aperture in each eye plate that allows efficient application of medications, and that is configured to be assembled and disassembled without using a tool.

According to an aspect of the present invention, a frame assembly for wearing on the head of a person comprises a frame front, the frame front including a left eye plate having a first opaque major surface defining a first optical aperture and a first liquid aperture; and a right eye plate, coupled to the left eye plate. The right eye plate has a second opaque major surface defining a second optical aperture and a second liquid aperture, the first optical aperture and the first liquid aperture defining a line transverse to a line defined by the first and second optical apertures, and the second optical aperture and the second liquid aperture defining a line transverse to the line defined by the first and second optical apertures. The frame front includes a first left front-engagement-structure; a second left front-engagement-structure; a first right front-engagement-structure; and a second right front-engagement-structure.

A left temple includes a first temple-engagement-structure configured to be removably engagable with the first left front-engagement-structure and a second temple-engagement-structure configured to be removably engagable with the second left front-engagement-structure; and a right temple including a first temple-engagement-structure and a second temple-engagement-structure, the right temple being subject to deformation force such that a distance E between the first and second temple-engagement-structures has a first inequality relation to a distance S between the first and second right front-engagement-structures, the right temple being resilient such that, after deformation ceases, the distance E has a second inequality relation to the distance S, the second inequality relation being an inverse of the first inequality relation, thereby enabling the right temple to be removably attachable to the frame front, without using a tool.

According to another aspect of the present invention, a frame assembly for wearing on the head of a person comprises a frame front, the frame front including a left eye plate having a first opaque major surface defining a first optical aperture and a first liquid aperture; a right eye plate, coupled to the left eye plate. The right eye plate has a second opaque major surface defining a second optical aperture and a second liquid aperture, the first optical aperture and the first liquid aperture defining a line transverse to a line defined by the first and second optical apertures, and the second optical aperture and the second liquid aperture defining a line transverse to the line defined by the first and second optical apertures. The frame front includes a first left surface defining a first left hole; a second left surface defining a second left hole; a first right surface defining a first right hole; a second right surface defining a second right hole. A left temple includes a first post configured to be removably engagable with the first left hole and a second post configured to be removably engagable with the second left hole; and a right temple includes a first post defining a first extremity and a second post defining a second extremity, the right temple being subject to deformation force such that a distance E between the first and second extremities has a first inequality relation to a distance S between the first and second right surfaces, the right temple being resilient such that, after deformation ceases, the distance E has a second inequality relation to the distance S, the second inequality relation being an inverse of the first inequality relation, thereby enabling the right temple to be removably attachable to the frame front, without using a tool.

According to yet another aspect of the present invention, these is a method for a frame assembly. The method comprises applying a first deformation force to the right temple such that a distance E between the first and second extremities has a first inequality relation to a distance S between the first and second right surfaces; subsequently, separating the right temple from the front part, thereby disassembling the frame assembly without using a tool; subsequently, ceasing applying the first deformation force such that the distance E has a second inequality relation to the distance S, the second inequality relation being an inverse of the first inequality relation; subsequently, subjecting the front part and the right temple to an antibacterial treatment; subsequently, applying a second deformation force to the right temple such that the distance E between the first and second extremities has a first inequality relation to the distance S between the first and second right surfaces; subsequently, bringing the right temple together with the front part; subsequently, ceasing applying the second deformation force such that the distance E has a second inequality relation to the distance S, the second inequality relation being an inverse of the first inequality relation, so as to engage the first post with the first right hole and the second post with the second right hole, thereby assembling the frame assembly without using a tool.

### BRIEF DESCRIPTION OF THE DRAWINGS

References are made, in the following text, to the accompanying drawings, in which:

FIG. 1 is a perspective view showing an exemplary device in use.

FIG. 2 is a rear perspective view showing the exemplary device.

FIG. 3 is a cross section side view of the exemplary device.

FIG. 4 is a side view of the exemplary device.

FIG. 5 is a cut view showing the exemplary device in assembled configuration.

FIG. 6 is a cut view showing the exemplary device in a transition between disassembly and assembly.

FIG. 7 is a cut view showing the exemplary device in disassembled configuration.

FIG. 8 is an enlarged drawing corresponding to FIG. 6 showing the exemplary device in transition between disassembly and assembly.

FIG. 9 is an enlarged drawing corresponding to FIG. 7 showing the exemplary device in disassembled configuration.

The accompanying drawings which are incorporated in and which constitute a part of this specification, illustrate embodiments of the invention and, together with the description, explain the principles of the invention, and additional advantages thereof. Certain drawings are not necessarily to scale, and certain features may be shown larger than relative actual size to facilitate a more clear description of those features. Throughout the drawings, corresponding elements are labeled with corresponding reference numbers.

### DETAILED DESCRIPTION OF EXEMPLARY EMBODIMENTS

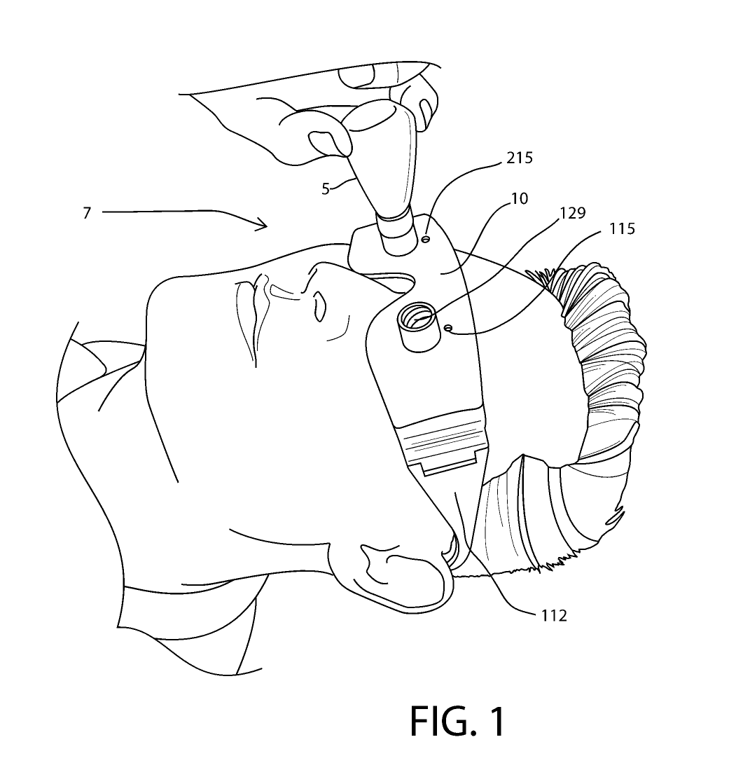

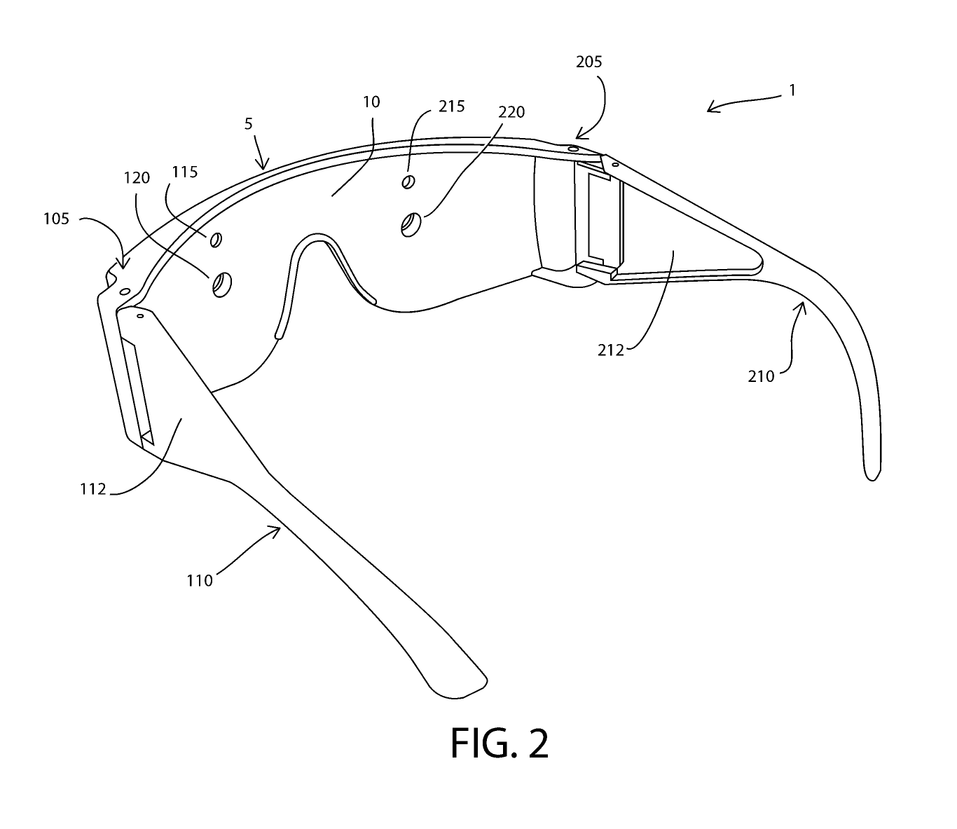

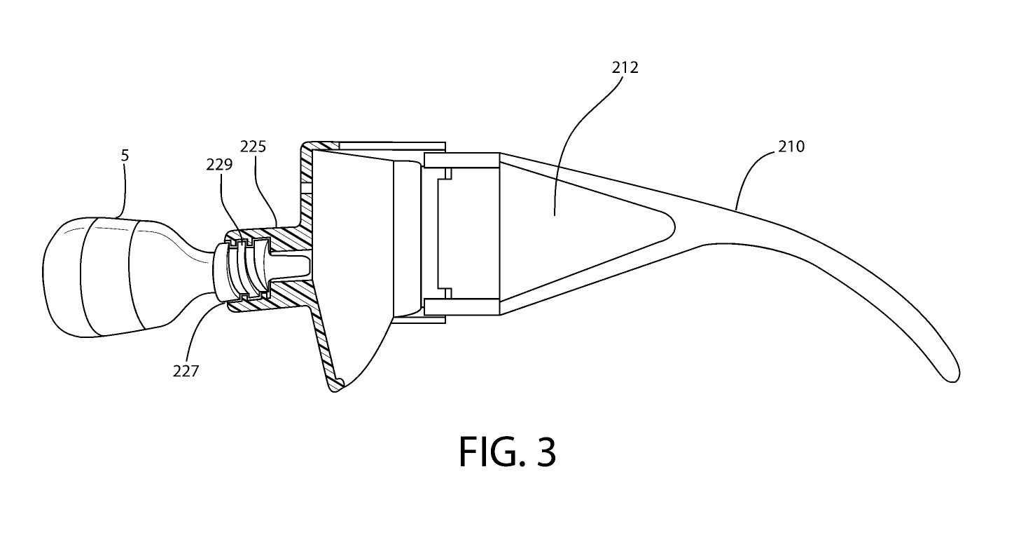

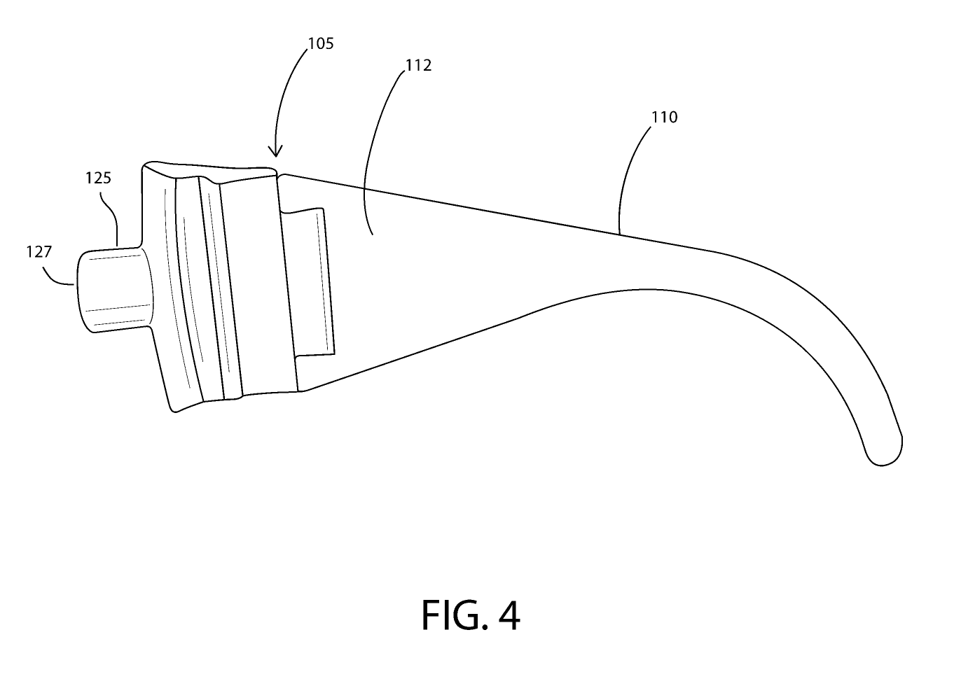

FIG. 1 shows a front perspective view of an exemplary frame assembly 1 according to a preferred embodiment of the present invention, and FIG. 2 is a back perspective view of frame assembly 1. Frame assembly 1 includes a front part 5 having an opaque major surface 10. A disassemblable hinge 105 couples a left temple 110 to the front part 5. The left temple 110 extends away from the opaque surface 10 in a direction transverse to the opaque surface 10, when the hinge 205 is in an open position.

A disassemblable hinge 205 couples a right temple 210 to the front part 5. Right temple 210 extends away from the opaque surface 10 in a direction transverse to the opaque surface 10, when the hinge 205 is in an open position.

The combination of left temple 110 and right temple 210 acts to secure eye front part 5 to the head of person 7.

The opaque surface 10 defines an optical aperture 115 and a liquid aperture 120. The liquid aperture 120 has a diameter of 5 mm, which is slightly wider than the tip of a typical eye drop bottle. The optical aperture 115 is a through hole defined by opaque surface 10. The optical aperture 115 and the liquid aperture 120 are separated by a distance of 8 millimeters. The optical aperture 115 and the liquid aperture 120 define a line transverse to a line defined by the optical aperture 115 and optical aperture 215.

The opaque surface 10 defines an optical aperture 215 and a liquid aperture 220. Liquid aperture 220 has a diameter of 5 mm, which is slightly wider than the tip of a typical eye drop bottle. The optical aperture 215 is a through hole defined by opaque surface 10. The optical aperture 215 and liquid aperture 220 are separated by a distance of 8 millimeters.

The optical aperture 115 is separated from the optical aperture 215 by a distance of 60 to 80 millimeters.

The left temple 110 defines an opaque surface 112 that acts to shield ambient light from the eyes of the user. The right temple 210 defines an opaque surface 212 that acts to shield ambient light from the eyes of the user.

Because the surfaces 10, 112 and 212 are opaque, optical aperture 115 and 215 are prominent optical targets for the wearer of frame assembly 1.

Each of bottle connectors 125 and 225 acts to support and align an eye drop bottle, such as bottle 5 shown in FIG. 1.

The bottle connector 125 has an end that is integral with opaque surface 10. The connector 125 defines an aperture 127 that is coaxial with the liquid aperture 120 of the surface 10. In other words, the end of connector 125 is integral with the surface 10 at an area around the liquid aperture 120. The connector 125 defines an inner, threaded surface 129.

The bottle connector 225 has an end that is integral with opaque surface 10. The connector 225 defines an aperture 227 that is coaxial with the liquid aperture 220 of the surface 10. In other words, the end of connector 225 is integral with the surface 10 at an area around liquid aperture 220. The connector 225 defines an inner, threaded surface 229.

Connector 125 is integrally formed with front part 5 and connector 225 is integrally formed with front part 5. This integral formation can be carried out with injection molding.

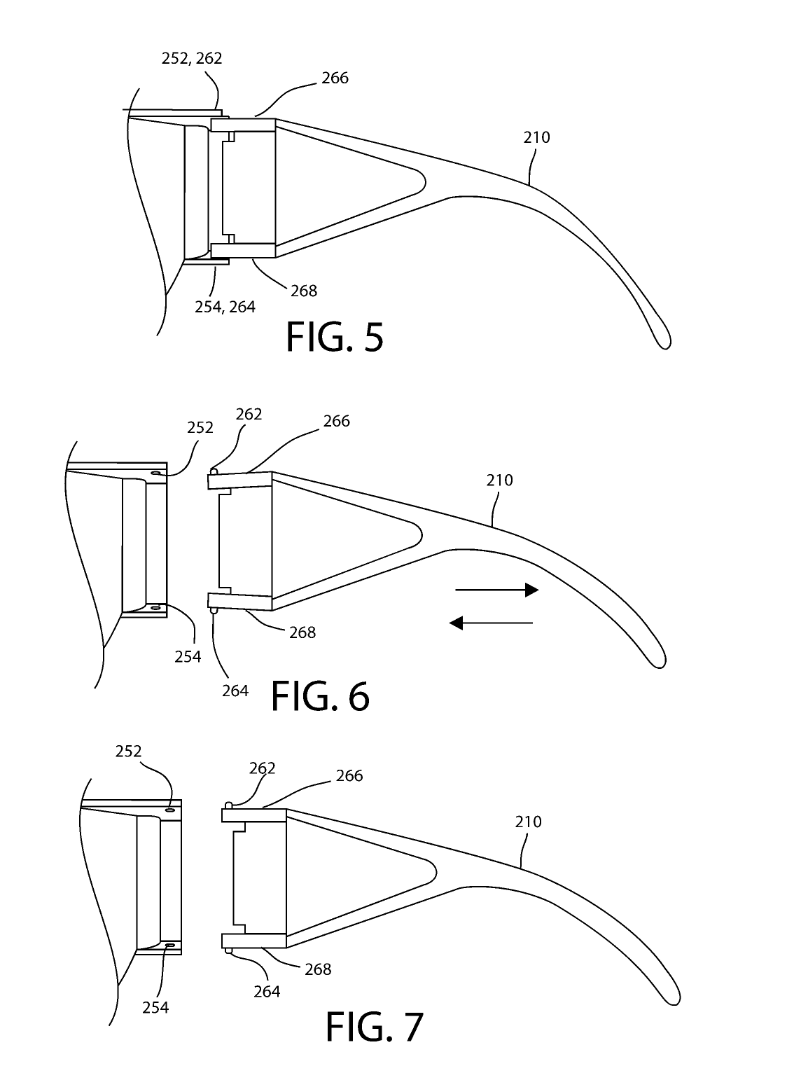

Referring to FIG. 6, a hole 252 is separated from the hole 254 by a distance S. The extensions 266 and 268 are compressible toward each other, using for example a thumb and forefinger. Subsequently, the post 262 is engaged with the hole 252, and the post 264 is engaged with the hole 254 via a recovery force of the extensions 266 and 268, so that the right temple 210 is connected to the front part 5.

Thus, attaching temple 210 to front part 5 includes the substep of compressing, during insertion of the right temple 210 into the front part 5, a such that the original shape of the right temple 210 can be recovered when parts 262 and 264 are in alignment with holes 252 and 254 respectively, such that a connection is established between the temple 210 and the front part 5.

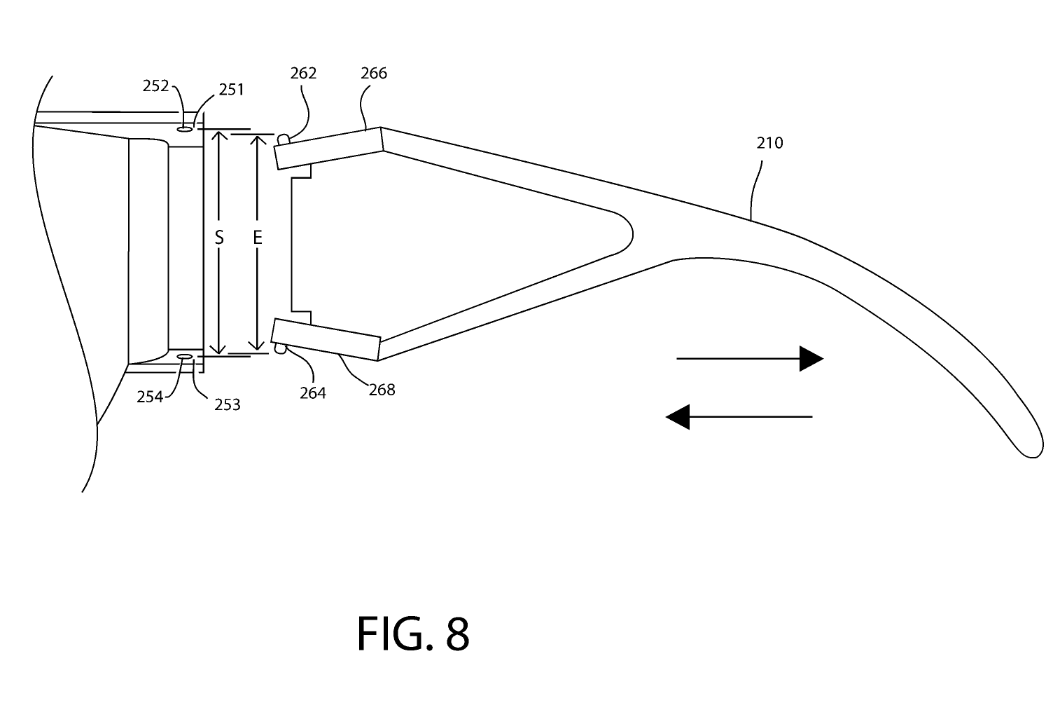

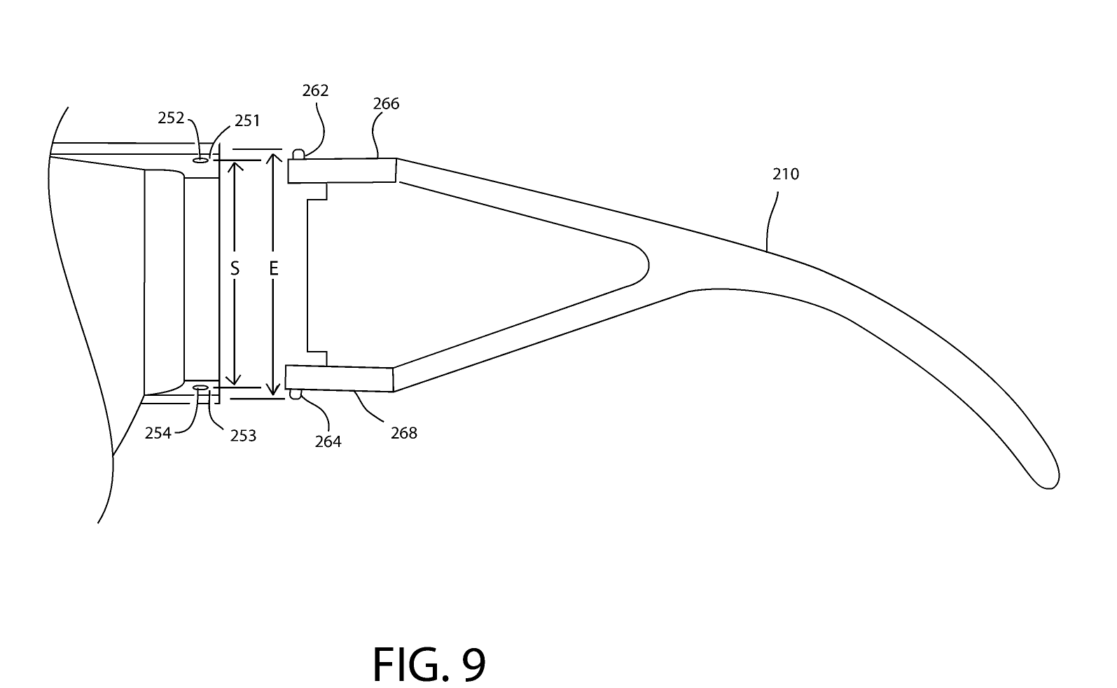

In other words, the frame assembly 1 is configured for wearing on the head of a person. The frame assembly 1 includes a frame front 5. The frame front includes a first right surface 251 (FIG. 8) defining a first right hole 252; and a second right surface 253 defining a second right hole 254. The right temple 210 includes a post 262 configured to be removably engagable with the hole 252 and a post 264 configured to be removably engagable with the hole 254. More specifically the post 262 defines a first extremity at a detail end of the post 262 and the post 264 defines a second extremity at a detail end of the post 264. The right temple 210 is subject to a compression force such that a distance E (FIG. 8 and FIG. 6) between the first and second extremities is less than a distance S between the surfaces 251 and 253. The right temple is resilient such that, after compression ceases, the distance E is greater than the distance S (FIG. 9, FIG. 7, and FIG. 5), thereby enabling the right temple 210 to be removably attachable to the frame front 5, without using a tool.

The left temple 110 includes a post 162 configured to be removably engagable with the hole 152 and a post 164 configured to be removably engagable with the hole 154. More specifically the post 162 defines a first extremity and the post 164 defines a second extremity. The left temple 110 is subject to a compression force such that a distance E between the first and second extremities is less that a distance S between the surfaces 151 and 153. The left temple is resilient such that, after compression ceases, the distance E is greater than the distance S.

Thus, the frame assemble 1 has snap back recovery, of the temples 110 and 210 in order to engage temples 110 and 210 with front part 5.

In other words, each of temples 110 and 210 is compressible so as to fit into the front part to engage holes.

In other words, to assemble the frame assembly 1, starting with a temple detached from front part 5, and in a relaxed position such that E>S, (FIGS. 7 and 9), use a thumb and finger to compress the temple such that E≦S (FIGS. 6 and 8); slide the temple into the front part 5, as indicated by the left arrow in FIGS. 6 and 8; and cease the compression, such that the resilience of the temple causes E>S thereby engaging the posts of the temple with the holes of the front part as shown in FIG. 5.

To apply eye drops using the assembled frame assembly 1, a person 7 puts on frame assembly 1 as one would put on a pair of eyeglasses, screws an eye drop bottle 5 into connector 225, tilts his or her head back, looks through optical aperture 215 with the right eye, and squeezes the eye drop bottle 5. Following this procedure causes a drop of a medication to fall into the lower corneal area of the left eye near the lower eye lid. The normal blinking reflex is reduced because the eye is oriented toward the optical aperture 215, not the liquid aperture 220. A corresponding procedure is then carryout out for the right eye.

Thus, the preferred frame assemblies allow accurate application of eye drops. The narrow liquid apertures in the eye plates act to space an eye drop bottle away from the eye, reducing the possibility of the dropper tip touching the eye, and therefore reducing the possibility of infection. Further, because the eye plate surfaces are opaque, reducing the blink reflex, the possibility of misapplication of the drops, with the resulting waste of medicine, is reduced.

To disassemble the frame assembly 1, starting with a temple attached to the front part 5, and in a relaxed position such that E>S, (FIG. 5), use a thumb and finger to compress the temple such that E≦S and slide the temple out of the front part 5, as indicated by the right arrow in FIGS. 6 and 8.

In accordance with a second exemplary embodiment of the present invention, the posts on the temple arms are arranged facing towards each other, instead of away from each other in the first embodiment described above. In this second embodiment, to engage the temple with the front part, the user pulls (applies a tensile force to) the temple extensions away from each other, slides the temple onto the front part, and then releases the tensile force, allowing the posts to engage into holes on the front part.

In other words, in accordance with the second exemplary embodiment, a right temple includes a first post defining a first extremity and a second post defining a second extremity, the right temple being subject to a tensile force such that a distance E between the first and second extremities is greater than a distance S between the first and second right surfaces, the right temple being resilient such that, after the tensile force ceases, the distance E is less than the distance S.

In other words, in accordance with the second exemplary embodiment, the temple is stretchable so as to fit over surface of the front part and engage holes defined by the surfaces of the front part.

In summary of the embodiments described above, a temple includes a first post defining a first extremity and a second post defining a second extremity, the temple being subject to deformation force such that a distance E between the first and second extremities has a first inequality relation to a distance S between the first and second right surfaces, the temple being resilient such that, after deformation ceases, the distance E has a second inequality relation to the distance S, the second inequality relation being an inverse of the first inequality relation.

In accordance with a third exemplary embodiment of the present invention, the temples have holes for engaging with posts on the front part. In other words, in accordance with the third exemplary embodiment, a temple defines a first surface defining a first hole; a second surface defining a second hole. The front part includes a first post defining a first extremity and a second post defining a second extremity. The right temple is subject to compression force such that is less than a distance S between the first and second surfaces is less than a distance E between the extremities. The temple is resilient such that, after compression ceases, the distance S is greater than the distance E.

In accordance with a variant embodiment, most of the frame assembly is made of soft silicone or silastic that can bend and twist.

Benefits, other advantages, and solutions to problems have been described above with regard to specific examples. The benefits, advantages, solutions to problems, and any element(s) that may cause any benefit, advantage, or solution to occur or become more pronounced are not critical, required, or essential feature or element of any of the claims.

Additional advantages and modifications will readily occur to those skilled in the art. The invention in its broader aspects is therefore not limited to the specific details, representative apparatus, and illustrative examples shown and described. Accordingly, departures may be made from such details without departing from the spirit or the scope of Applicants' general inventive concept. The invention is defined in the following claims. In general, the words “first,” “second,” etc., employed in the claims do not necessarily denote an order.

What is claimed is:

1. A frame assembly for wearing on the head of a person, the frame assembly comprising
a frame front, the frame front including
a left eye plate having a first opaque major surface defining a first optical aperture and a first liquid aperture;
a right eye plate, coupled to the left eye plate, having a second opaque major surface defining a second optical aperture and a second liquid aperture, the first optical aperture and the first liquid aperture defining a line transverse to a line defined by the first and second optical apertures, and the second optical aperture and the second liquid aperture defining a line transverse to the line defined by the first and second optical apertures;
a first left front-engagement-structure;
a second left front-engagement-structure;
a first right front-engagement-structure;
a second right front-engagement-structure;
a left temple including a first temple-engagement-structure configured to be removably engagable with the first left front-engagement-structure and a second temple-engagement-structure configured to be removably engagable with the second left front-engagement-structure; and
a right temple including a first temple-engagement-structure and a second temple-engagement-structure, the right temple being subject to deformation force such that a distance E between the first and second temple-engagement-structures has a first inequality relation to a distance S between the first and second right front-engagement-structures, the right temple being resilient such that, after deformation ceases, the distance E has a second inequality relation to the distance S, the second inequality relation being an inverse of the first inequality relation, thereby enabling the right temple to be removably attachable to the frame front, without using a tool.

2. A frame assembly according to claim 1 wherein the deformation force is a compression force, the first inequality relation is E≦S, and the second inequality relation is E>S.

3. A frame assembly according to claim 1 wherein the deformation force is a tension force, the first inequality relation is E≧S, and the second inequality relation is E<S.

4. A frame assembly according to claim 1 wherein the front-engagement structures are female and the temple-engagement structures are male.

5. A frame assembly according to claim 4 wherein the deformation force is a compression force, the first inequality relation is E≦S, and the second inequality relation is E>S.

6. A frame assembly according to claim 4 wherein the deformation force is a tension force, the first inequality relation is E≧S, and the second inequality relation is E<S.

7. A frame assembly according to claim 1 wherein the front-engagement structures are holes and the temple-engagement structures are posts.

8. A frame assembly according to claim 7 wherein the deformation force is a compression force, the first inequality relation is E≦S, and the second inequality relation is E>S.

9. A frame assembly according to claim 7 wherein the deformation force is a tension force, the first inequality relation is E≧S, and the second inequality relation is E<S.

10. A frame assembly for wearing on the head of a person, the frame assembly comprising
a frame front, the frame front including
a left eye plate having a first opaque major surface defining a first optical aperture and a first liquid aperture;
a right eye plate, coupled to the left eye plate, having a second opaque major surface defining a second optical aperture and a second liquid aperture, the first optical aperture and the first liquid aperture defining a line transverse to a line defined by the first and second optical apertures, and the second optical aperture and the second liquid aperture defining a line transverse to the line defined by the first and second optical apertures;
a first left surface defining a first left hole;
a second left surface defining a second left hole;
a first right surface defining a first right hole;
a second right surface defining a second right hole;
a left temple including a first post configured to be removably engagable with the first left hole and a second post configured to be removably engagable with the second left hole; and
a right temple including a first post defining a first extremity and a second post defining a second extremity, the right temple being subject to deformation force such that a distance E between the first and second extremities has a first inequality relation to a distance S between the first and second right surfaces, the right temple being resilient such that, after deformation ceases, the distance E has a second inequality relation to the distance S, the second inequality relation being an inverse of the first inequality relation, thereby enabling the right temple to be removably attachable to the frame front, without using a tool.

11. A frame assembly according to claim 10 wherein the deformation force is a compression force, the first inequality relation is E≦S, and the second inequality relation is E>S.

12. A frame assembly according to claim 10 wherein the deformation force is a tension force, the first inequality relation is E≧S, and the second inequality relation is E<S.

13. A frame assembly according to claim 10 wherein most of the frame assembly is made of soft silicone or silastic that can bend and twist.

14. A method for a frame assembly, the frame assembly including
a frame front, the frame front including
a left eye plate having a first opaque major surface defining a first optical aperture and a first liquid aperture;
a right eye plate, coupled to the left eye plate, having a second opaque major surface defining a second optical aperture and a second liquid aperture, the first optical aperture and the first liquid aperture defining a line transverse to a line defined by the first and second optical apertures, and the second optical aperture and the second liquid aperture defining a line transverse to the line defined by the first and second optical apertures;
a first left surface defining a first left hole;
a second left surface defining a second left hole;
a first right surface defining a first right hole;
a second right surface defining a second right hole;
a right temple including a first post defining a first extremity and a second post defining a second extremity, the method comprising:
applying a first deformation force to the right temple such that a distance E between the first and second extremities has a first inequality relation to a distance S between the first and second right surfaces;
subsequently, separating the right temple from the front part, thereby disassembling the frame assembly without using a tool;
subsequently, ceasing applying the first deformation force such that the distance E has a second inequality relation to the distance S, the second inequality relation being an inverse of the first inequality relation;
subsequently, subjecting the front part and the right temple to an antibacterial treatment;
subsequently, applying a second deformation force to the right temple such that the distance E between the first and second extremities has a first inequality relation to the distance S between the first and second right surfaces;
subsequently, bringing the right temple together with the front part;
subsequently, ceasing applying the second deformation force such that the distance E has a second inequality relation to the distance S, the second inequality relation being an inverse of the first inequality relation, so as to engage the first post with the first right hole and the second post with the second right hole, thereby assembling the frame assembly without using a tool.

15. A method according to claim 14 wherein each of the first and second deformation forces is a compression force, the first inequality relation is E≦S, and the second inequality relation is E>S.

16. A method according to claim 14 wherein each of the first and second deformation forces deformation force is a tension force, the first inequality relation is E≧S, and the second inequality relation is E<S.

17. A method according to claim 14 wherein subjecting the front part and the right temple to an antibacterial treatment includes washing the front part and the right temple with soap and water.
